(19) United States
(12) Patent Application Publication
(10) Pub. No.: US 2011/0300228 A1
(43) Pub. Date: Dec. 8, 2011

(54) COMPOSITION, WITH/WITHOUT AT LEAST ONE EASILY OXIDISED COMPONENT, COMPRISING AN EDIBLE PROCESSED OIL OR FAT MIXTURE, FOR PROMOTING GROWTH IN AN ANIMAL, PREVENTING OXIDATIVE STRESS, AVOIDING THAT FISH DEVELOP ENLARGED LIVER AND FEED COMPOSITION

(75) Inventors: Harald Breivik, Porsgrunn (NO); Olav Thorstad, Porsgrunn (NO); Bente Synnøve Ruyter, As (NO)

(73) Assignee: PRONOVA BIOPHARMA NORGE AS, Baerum (NO)

(21) Appl. No.: 13/131,625

(22) PCT Filed: Nov. 27, 2009
(86) PCT No.: PCT/NO2009/000410

§ 371 Date: Aug. 11, 2011

Related U.S. Application Data
(60) Provisional application No. 61/118,495, filed on Nov. 28, 2008.

(51) Int. Cl. A61K 35/60; A61P 39/06; A61P 15/00

(52) U.S. Cl. 424/523

(57) ABSTRACT

The present invention relates to a composition for reducing the tendency of an animal to enter sexual maturation, reducing or preventing oxidative damage in an animal, for reducing or preventing oxidative stress in an animal, promoting growth of an animal and for avoiding enlarged liver size in fish. The present invention also relates to a composition having improved characteristics with regard to the stability of easily oxidised components during production and storage; and with regard to the utilization of the easily oxidised components.

The present invention relates to a composition for reducing the tendency of an animal to enter sexual maturation, reducing or preventing oxidative damage in an animal, for reducing or preventing oxidative stress in an animal, promoting growth of an animal and for avoiding enlarged liver size in fish. The present invention also relates to a composition having improved characteristics with regard to the stability of easily oxidised components during production and storage; and with regard to the utilization of the easily oxidised components.

### BACKGROUND OF THE INVENTION

There are numerous publications on the unique nutritional and health benefits, to both humans and animals, from eating fish and consuming seafood derived oils containing omega-3 fatty acids. Not only is fish an unrivaled protein source, but its content of the beneficial omega-3 fatty acids has been shown to play a vital role in preventing heart disease, as well as benefiting the immune and nervous systems and assisting against a wide range of disorders.

The well known health benefits from eating fish and consuming seafood derived oils containing omega-3 fatty acids have turned health conscious consumers from meat to fish. However, in face of increasingly threatened wild fish stocks, the catch of wild fish will have to be reduced in order for the fish to be sustainable.

One solution to the supply problem is to grow the fish in fish farms where conditions are controlled and ecological conditions are not upset by overfishing. Fish farming has brought prices down and made fish supplies steadier and more dependable.

However, it has turned out that farm-raised fish may not be as healthy as presumed at first. Extensive analysis have indicated that many of the farm-raised fish has significantly higher dioxin levels than those found in wild fish. Most likely, these pollutants accumulated in fish processed into the farm fish feed due to environmental pollution and are recognized to have a significant adverse effect on human health. Consequently, removal or reduction of pollutants in food/feed products have the potential to substantially increase marketability and value.

From the literature it is known that molecular distillation, or short path distillation as the technique alternatively may be named, can be used to remove the pesticides DDT and its metabolites from fish oil (K. Julshamn, L. Karlsen and O. R. Braekkan, Removal of DDT and its metabolites from fish oils by molecular distillation, Fiskeridirektoratets skrifter; Serie teknologiske undersøkelser, Vol. 5 No. 15 (1973)). A practical upper limit was 65% removal together with a loss of about 25% of vitamin A. In many industrial fish oil refining processes a removal of DDT up to 65% is not satisfactory.

Anthony P. Bimbo: Guidelines for characterization of food-grade fish oil. INFORM 9(5), 473-483 (1998), reported that vacuum stripping or thin-film distillation can be used to remove chlorinated hydrocarbons and free fatty acids from fats or oils. A disadvantage by using vacuum stripping to refine oils is that sufficient results only can be achieved then the vacuum stripping process is carried out at a high temperature which may give rise to undesirable side reactions.

Jiri Cmolik og Jan Pokorny: Physical refining of edible oils, Eur. J. Lipid Sci. Technol. 102(7), 472-486 (2000) describes physical refining of edible oils and the use of molecular distillation for removal of undesirable substances in crude oils, preferably crude vegetable oils, respectively the use of steam stripping in order to remove free fatty acids from an oil composition. Physical refining is used to refine oils of good quality, i.e. oils with small amounts of free fatty acids. However, physical refining is more complicated and costly for oils with inferior quality.

In WO 9524459 a process for treating an oil composition containing saturated and unsaturated fatty acids in the form of triglycerides, in order to obtain a refined product with higher concentrations of the polyunsaturated fatty acids, is presented. This process also is intended to be used for removal of some environmental pollutants from an oil composition, wherein the process comprises the steps of subjecting the oil composition to a transesterification reaction and thereafter subjecting the product obtained in the first step to one or more molecular distillations. This technique has the severe limitation that it can only be used for fish oils that have been partially transesterified using a lipase catalyst that discriminates against omega-3 fatty acids. Obviously, this technique can not be used for commercial fish oils.

In EP0632267 a method of measuring the content of polycyclic aromatic hydrocarbons (PAH) remaining in lanolin is presented. The European patent document also describes a method of removing PAH remaining in wool grease or lanolin by a vacuum distillation of the grease or lanolin under specified conditions either directly or after having been treated with a borate and, if necessary, obtaining various lanolin derivatives from the treated wool grease or lanolin. However, the technique described in said patent document requires very high temperatures (230° C.) in order to achieve 90% reduction in PAH content.

WO2004/007654 discloses a process for decreasing the amount of environmental pollutants in a fat or an oil. The disclosed process involves adding a volatile working fluid to an oil or fat mixture, and then subjecting the oil or fat mixture with the added volatile working fluid to at least one stripping processing step, in which an amount of environmental pollutant present in the fat or oil is separated from the mixture together with the volatile working fluid. The end-product of the process disclosed in WO2004/007654 is a high quality oil almost free from environmental pollutants.

The object of the present invention is to identify new uses of the end-product of the process disclosed in WO2004/007654.

### SUMMARY OF THE INVENTION

A first aspect of the present invention relates to a composition comprising an edible processed oil or fat mixture for reducing the tendency of an animal to enter sexual maturation, said processed oil or fat mixture being obtainable by a method comprising the following steps:
providing a mixture comprising oil or fat, said oil or fat containing environmental pollutants;
subjecting the mixture to at least one stripping processing step, in which an amount of environmental pollutant present in the mixture is separated from the mixture.

A second aspect of the present invention relates to a composition comprising an edible processed oil or fat mixture for reducing or preventing oxidative damage in an animal, said processed oil or fat mixture being obtainable by a method comprising the following steps:
providing a mixture comprising oil or fat, said oil or fat containing environmental pollutants;
subjecting the mixture to at least one stripping processing step, in which an amount of environmental pollutant present in the mixture is separated from the mixture.

A third aspect of the present invention relates to a composition comprising an edible processed oil or fat mixture for reducing or preventing oxidative stress in an animal, said processed oil or fat mixture being obtainable by a method comprising the following steps:
providing a mixture comprising oil or fat, said oil or fat containing environmental pollutants;
subjecting the mixture to at least one stripping processing step, in which an amount of environmental pollutant present in the mixture is separated from the mixture.

A forth aspect of the present invention relates to a composition comprising an edible processed oil or fat mixture and at least one easily oxidised component, said processed oil or fat mixture being obtainable by a method comprising the following steps:
providing a mixture comprising oil or fat, said oil or fat containing environmental pollutants;
subjecting the mixture to at least one stripping processing step, in which an amount of environmental pollutant present in the mixture is separated from the mixture.

A fifth aspect of the present invention relates to a composition comprising an edible processed oil or fat mixture, said processed oil or fat mixture being obtainable by a method comprising the following steps:
providing a mixture comprising oil or fat, said oil or fat containing environmental pollutants;
subjecting the mixture to at least one stripping processing step, in which an amount of environmental pollutant present in the mixture is separated from the mixture;
   for promoting growth of a non-human animal beyond what is achieved when using a composition comprising said oil or fat mixture that has not been subjected to said processing steps.

A further aspect of the present invention relates to a composition comprising an edible processed oil or fat mixture, said processed oil or fat mixture being obtainable by a method comprising the following steps:
providing a mixture comprising oil or fat, said oil or fat containing environmental pollutants;
subjecting the mixture to at least one stripping processing step, in which an amount of environmental pollutant present in the mixture is separated from the mixture;
   for avoiding that fish develop enlarged liver.

Another aspect of the present invention relates to a feed composition comprising an edible processed oil or fat mixture, said processed oil or fat mixture being obtainable by a method comprising the following steps:
providing a mixture comprising oil or fat, said oil or fat containing environmental pollutants;
subjecting the mixture to at least one stripping processing step, in which an amount of environmental pollutant present in the mixture is separated from the mixture.

Preferred embodiments of the present invention are set forth in the dependent claims.

### DESCRIPTION OF THE FIGURES

FIG. 1

Illustration of a molecular distillation plant.

### DETAILED DESCRIPTION OF THE INVENTION

Sexual maturation of fanned fish is undesirable. This gives the fish reduced appetite, with a reduced weight increase except for the commercially undesirable growth of the gonads. Also, the quality of the fish will be reduced. For salmon, mature fish have practically no sales value.

Surprisingly, it has been discovered that a fish feed comprising an edible oil or fat mixture which has been subjected to a method comprising the following steps:
providing a mixture comprising oil or fat, said oil or fat containing environmental pollutants;
subjecting the mixture to at least one stripping processing step, in which an amount of environmental pollutant present in the mixture is separated from the mixture;
   reduces the tendency of a fish to enter sexual maturation compared with fish that has been given a feed composition comprising said oil or fat mixture that has not been subjected to said processing steps (see example 4).

Thus, a first aspect of the present invention relates to a composition comprising an edible processed oil or fat mixture for reducing the tendency of an animal to enter sexual maturation, said processed oil or fat mixture being obtainable by a method comprising the following steps:
providing a mixture comprising oil or fat, said oil or fat containing environmental pollutants;
subjecting the mixture to at least one stripping processing step, in which an amount of environmental pollutant present in the mixture is separated from the mixture.

In one preferred embodiment according to the first aspect of the present invention, the number of fish that have entered sexual maturation at a weight of around 3 kg is reduced by at least 15%, more preferably reduced by at least 20% even more preferably reduced by at least 25% if the fish are fed a feed comprising said processed oil or fat mixture as compared to fish fed a feed comprising said non-processed oil or fat mixture.

Further, it has been found that a fish feed comprising an edible oil or fat mixture which has been subjected to a method comprising the following steps:
providing a mixture comprising oil or fat, said oil or fat containing environmental pollutants;
subjecting the mixture to at least one stripping processing step, in which an amount of environmental pollutant present in the mixture is separated from the mixture;
   reduces oxidative damage in fish compared with fish that has been given a feed composition comprising said oil or fat mixture that has not been subjected to said processing steps (see example 3).

Documentary evidence of increased growth and reduced oxidative damage, as shown in example 3 and 2B, indicate that the fish feed comprising said processed oil or fat mixture gives the farmed fish better health compared with fish fed a similar composition, where said oil or fat mixture has not been subjected to said processing steps.

Thus, a second aspect of the present invention relates to a composition comprising an edible processed oil or fat mixture for reducing or preventing oxidative damage in an animal, said processed oil or fat mixture being obtainable by a method comprising the following steps:
providing a mixture comprising oil or fat, said oil or fat containing environmental pollutants;
subjecting the mixture to at least one stripping processing step, in which an amount of environmental pollutant present in the mixture is separated from the mixture.

In a preferred embodiment according to the second aspect of the present invention, said composition is for non-therapeutic reduction or prevention of oxidative damage in an animal.

Most reactive oxygen species are produced at a low level by normal aerobic metabolism and the damage they cause to cells is constantly repaired (reversible damage). Thus, non-therapeutic reduction of oxidative damage is herein defined as reduction of reversible oxidative damage, i.e. reduction of repairable oxidative damage.

It has also been found that a fish feed comprising an edible oil or fat mixture which has been subjected to a method comprising the following steps:
providing a mixture comprising oil or fat, said oil or fat containing environmental pollutants;
subjecting the mixture to at least one stripping processing step, in which an amount of environmental pollutant present in the mixture is separated from the mixture;
   reduces oxidative stress in fish compared with fish that has been given a feed composition comprising said oil or fat mixture that has not been subjected to said processing steps.

Oxidative stress is caused by an imbalance between the production of reactive oxygen and a biological system's ability to readily detoxify the reactive intermediates or easily repair the resulting damage. All forms of life maintain a reducing environment within their cells. This reducing environment is preserved by enzymes that maintain the reduced state through a constant input of metabolic energy. Disturbances in this normal redox state can cause toxic effects through the production of peroxides and free radicals that damage all components of the cell, including proteins, lipids, and DNA.

Thus, a thirds aspect of the present invention relates to a composition comprising an edible processed oil or fat mixture for reducing or preventing oxidative stress in an animal, said processed oil or fat mixture being obtainable by a method comprising the following steps:
providing a mixture comprising oil or fat, said oil or fat containing environmental pollutants;
subjecting the mixture to at least one stripping processing step, in which an amount of environmental pollutant present in the mixture is separated from the mixture.

In a preferred embodiment according to the third aspect of the present invention, said composition is for non-therapeutic reduction or prevention of oxidative stress in an animal.

Most reactive oxygen species are produced at a low level by normal aerobic metabolism and the damage they cause to cells is constantly repaired (reversible damage). Thus, non-therapeutic reduction of oxidative damage is herein defined as reduction of reversible oxidative damage, i.e. reduction of repairable oxidative damage.

It was also surprisingly discovered that the loss of astaxanthin, during production and storage of fish feed, is significantly reduced if the oil or fat mixture in said fish feed is subjected to a method comprising the following steps:
providing a mixture comprising oil or fat, said oil or fat containing environmental pollutants;
subjecting the mixture to at least one stripping processing step, in which an amount of environmental pollutant present in the mixture is separated from the mixture (example 2A).

It was also discovered that the use of an oil or fat mixture, which has been subjected to a method comprising the following steps:
providing a mixture comprising oil or fat, said oil or fat containing environmental pollutants;
subjecting the mixture to at least one stripping processing step, in which an amount of environmental pollutant present in the mixture is separated from the mixture,
   in a fish feed containing astaxanthin resulted in a significant better utilisation of the expensive astaxanthin pigment as compared to fish feed comprising astaxanthin and the non-processed oil or fat mixture (oil or fat mixture which has not been subjected to said processing steps) (example 2B).

Thus, a fourth aspect of the present invention relates to a composition comprising an edible processed oil or fat mixture and at least one easily oxidised component, said processed oil or fat mixture being obtainable by a method comprising the following steps:
providing a mixture comprising oil or fat, said oil or fat containing environmental pollutants;
subjecting the mixture to at least one stripping processing step, in which an amount of environmental pollutant present in the mixture is separated from the mixture.

In one embodiment according to the fourth aspect of the present invention, said at least one easily oxidised component has less tendency to oxidise during production and storage if the composition comprises said processed oil or fat mixture than if the composition comprises the non-processed oil or fat mixture.

In another embodiment according to the fourth aspect of the present invention, the utilization of said at least one easily oxidised component is increased in a composition comprising said processed oil or fat mixture as compared to what is the case for a composition comprising the non-processed oil or fat mixture.

Antioxidants are examples of easily oxidised components. Antioxidants are oxidised in order to prevent oxidation of other components in the composition. Astaxanthin, tocopherols, tocotrienols, ubiquinones and lutein are examples of natural antioxidants. Polyunsaturated fatty acids and derivatives of polyunsaturated fatty acids are also examples of easily oxidised components.

In a further embodiment according to the fourth aspect of the present invention, said composition is a health supplement. As used herein the term health supplement is interpreted to include food and food supplement to animals and/or humans, fortification of food, dietary supplement, functional (and medical) food and nutrient supplement.

Further, it was discovered that a fish feed comprising an edible oil or fat mixture which has been subjected to a method comprising the following steps:
providing a mixture comprising oil or fat, said oil or fat containing environmental pollutants;
subjecting the mixture to at least one stripping processing step, in which an amount of environmental pollutant present in the mixture is separated from the mixture;
   promotes growth of fish beyond what is achieved when using a feed composition comprising said oil or fat mixture that has not been subjected to said processing steps. Said growth promoting effect is achieved without affecting the muscle structure negatively (example 7B).

When the feeding study started, there was no indication that producing fish with reduced levels of environmental pollutants would give better fish growth. On the contrary, there was an awareness that cleaning of environmental pollutants from the feed oils would lead to reduced amounts of components that would be beneficial for the growth and health of the fish. The study was initiated in order to document whether there could be any negative effects of using cleaned feed oil. Surprisingly the fish receiving oil cleaned with regards to environmental pollutants also had better growth than the control group (see example 3 and 2B).

Thus, a fifth aspect of the present invention relates to a composition comprising an edible processed oil or fat mixture, said processed oil or fat mixture being obtainable by a method comprising the following steps:
providing a mixture comprising oil or fat, said oil or fat containing environmental pollutants;
subjecting the mixture to at least one stripping processing step, in which an amount of environmental pollutant present in the mixture is separated from the mixture;
   for promoting growth of a non-human animal beyond what is achieved when using a composition comprising said oil or fat mixture that has not been subjected to said processing steps.

In one embodiment according to the present invention, the growth promoting effect is achieved without negatively affecting the muscle structure.

Further, it has been discovered that a fish feed comprising an edible oil or fat mixture which has been subjected to a method comprising the following steps:
providing a mixture comprising oil or fat, said oil or fat containing environmental pollutants;
subjecting the mixture to at least one stripping processing step, in which an amount of environmental pollutant present in the mixture is separated from the mixture;
   avoids that fish develop enlarged liver (example 6).

For some farmed fish species, i.e. cod, pollock, saithe and coalfish enlarged liver size compared to wild fish is a significant economical problem, as much of the feed is just utilised for increasing the liver size. Typically, the hepatosomatic index for farmed cod can be above 10%. Thus, purification of feed oil according to the present invention will be of value for improving the economy of the fish farming industry by reducing the amount of feed that is utilised for producing enlarged fish livers.

Thus, a further aspect of the present invention relates to a composition comprising an edible processed oil or fat mixture, said processed oil or fat mixture being obtainable by a method comprising the following steps:
providing a mixture comprising oil or fat, said oil or fat containing environmental pollutants;
subjecting the mixture to at least one stripping processing step, in which an amount of environmental pollutant present in the mixture is separated from the mixture;
   for avoiding that animals, particularly fish, such as cod, pollock, saithe and coalfish, develop enlarged liver.

In a preferred embodiment, the hepatosomatic index for animals that has been given a composition comprising said processed oil or fat mixture is lower than what is the case for animals that has been given the composition comprising the non-processed oil or fat mixture.

A sixth aspect of the present invention relates to the use of a composition comprising an edible processed oil or fat mixture for reducing the tendency of an animal to enter sexual maturation, said processed oil or fat mixture being obtainable by a method comprising the following steps:
providing a mixture comprising oil or fat, said oil or fat containing environmental pollutants;
subjecting the mixture to at least one stripping processing step, in which an amount of environmental pollutant present in the mixture is separated from the mixture.

In one preferred embodiment according to the sixth aspect of the present invention, the number of fish that have entered sexual maturation at a weight of around 3 kg is reduced by at least 15%, more preferably reduced by at least 20% even more preferably reduced by at least 25% if the fish are fed a feed comprising said processed oil or fat mixture as compared to fish fed a feed comprising said non-processed oil or fat mixture.

A seventh aspect of the present invention relates to the use of a composition comprising an edible processed oil or fat mixture for reducing or preventing oxidative damage in an animal, said processed oil or fat mixture being obtainable by a method comprising the following steps:
providing a mixture comprising oil or fat, said oil or fat containing environmental pollutants;
subjecting the mixture to at least one stripping processing step, in which an amount of environmental pollutant present in the mixture is separated from the mixture.

In a preferred embodiment according to the seventh aspect of the present invention, said use is for non-therapeutic reduction or prevention of oxidative damage in an animal.

Most reactive oxygen species are produced at a low level by normal aerobic metabolism and the damage they cause to cells is constantly repaired (reversible damage). Thus, non-therapeutic reduction of oxidative damage is herein defined as reduction of reversible oxidative damage, i.e. reduction of repairable oxidative damage.

An eighth aspect of the present invention relates to the use of a composition comprising an edible processed oil or fat mixture for reducing or preventing oxidative stress in an animal, said processed oil or fat mixture being obtainable by a method comprising the following steps:
providing a mixture comprising oil or fat, said oil or fat containing environmental pollutants;
subjecting the mixture to at least one stripping processing step, in which an amount of environmental pollutant present in the mixture is separated from the mixture.

In a preferred embodiment according to the third aspect of the present invention, said use is for non-therapeutic reduction or prevention of oxidative stress in an animal.

Most reactive oxygen species are produced at a low level by normal aerobic metabolism and the damage they cause to cells is constantly repaired (reversible damage). Thus, non-therapeutic reduction of oxidative damage is herein defined as reduction of reversible oxidative damage, i.e. reduction of repairable oxidative damage.

A ninth aspect of the present invention relates to the use of an edible processed oil or fat mixture in a composition comprising at least one easily oxidised component, e.g. astaxanthin, said processed oil or fat mixture being obtainable by a method comprising the following steps:
providing a mixture comprising oil or fat, said oil or fat containing environmental pollutants;
subjecting the mixture to at least one stripping processing step, in which an amount of environmental pollutant present in the mixture is separated from the mixture.

In one embodiment according to the ninth aspect of the present invention, said at least one easily oxidised component has less tendency to oxidise during production and storage if the composition comprises said processed oil or fat mixture than if the composition comprises the non-processed oil or fat mixture.

In a further embodiment according to the ninth aspect of the present invention, said use is for reducing or preventing oxidation of said at least one easily oxidised component during production and storage.

In another embodiment according to the ninth aspect of the present invention, the utilization of said at least one easily oxidised component is increased in a composition comprising said processed oil or fat mixture as compared to what is the case for a composition comprising the non-processed oil or fat mixture.

In a further embodiment according to the ninth aspect of the present invention, said use is for increasing the utilization of said at least one easily oxidised component.

Antioxidants are examples of easily oxidised components. Antioxidants are oxidised in order to prevent oxidation of other components in the composition. Astaxanthin, tocopherols, tocotrienols, ubiquinones and lutein are examples of natural antioxidants. Polyunsaturated fatty acids and derivatives of polyunsaturated fatty acids are also examples of easily oxidised components.

In a further embodiment according to the ninth aspect of the present invention, said composition is a health supplement.

As used herein the term health supplement is interpreted to include food and food supplement to animals and/or humans, fortification of food, dietary supplement, functional (and medical) food and nutrient supplement.

A tenth aspect of the present invention relates to the use of a composition comprising an edible processed oil or fat mixture, said processed oil or fat mixture being obtainable by a method comprising the following steps:
providing a mixture comprising oil or fat, said oil or fat containing environmental pollutants;
subjecting the mixture to at least one stripping processing step, in which an amount of environmental pollutant present in the mixture is separated from the mixture;
   for promoting growth of a non-human animal beyond what is achieved when using a composition comprising said oil or fat mixture that has not been subjected to said processing steps.

In one embodiment according to the present invention, the growth promoting effect is achieved without negatively affecting the muscle structure.

Further, it has also been surprisingly discovered that despite the fact that processed oil or fat mixture being obtainable by a method comprising the following steps:
providing a mixture comprising oil or fat, said oil or fat containing environmental pollutants;
subjecting the mixture to at least one stripping processing step, in which an amount of environmental pollutant present in the mixture is separated from the mixture;
   seemed to contain small reduction of EPA and DHA as compared with the oil or fat mixture that has not been subjected to said processing steps, fish that had received feed with processed oil had fillets with higher content of EPA, DHA and total omega-3 fatty acids than fillets of fish that had received the same feed oil, but without purification (example 5).

Thus, an eleventh aspect of the present invention relates to the use of an edible omega-3 fatty acid containing processed oil or fat mixture in a composition, said processed oil or fat mixture being obtainable by a method comprising the following steps:
providing a mixture comprising oil or fat, said oil or fat containing environmental pollutants;
subjecting the mixture to at least one stripping processing step, in which an amount of environmental pollutant present in the mixture is separated from the mixture.
   for increasing the bioavailability of said omega-3 fatty acids and/or improve the utilization of said omega-3 fatty acids.

A twelfth aspect of the present invention relates to the use of an edible processed oil or fat mixture in a composition, said processed oil or fat mixture being obtainable by a method comprising the following steps:
providing a mixture comprising oil or fat, said oil or fat containing environmental pollutants;
subjecting the mixture to at least one stripping processing step, in which an amount of environmental pollutant present in the mixture is separated from the mixture;
   for avoiding that animals, particularly fish such as cod, pollock, saithe and coalfish develop enlarged liver.

A thirteenth aspect of the present invention relates to a feed composition comprising an edible processed oil or fat mixture, said processed oil or fat mixture being obtainable by a method comprising the following steps:
providing a mixture comprising oil or fat, said oil or fat containing environmental pollutants;
subjecting the mixture to at least one stripping processing step, in which an amount of environmental pollutant present in the mixture is separated from the mixture.

In a preferred embodiment according to the present invention, said composition is for reducing the tendency of a non-human animal, more preferably a marine animal such as fish, e.g. salmon or trout, to enter sexual maturation.

In another embodiment according to the present invention, said composition is for reducing or preventing oxidative damage in a non-human animal, more preferably a marine animal such as fish, e.g. salmon or trout.

In another embodiment according to the present invention, said composition is for reducing or preventing oxidative stress in a non-human animal, more preferably a marine animal such as fish, e.g. salmon or trout.

In another embodiment according to the present invention, said composition is for promoting growth of a non-human animal, more preferably a marine animal such as fish, e.g. salmon or trout, beyond what is achieved when using a composition comprising said oil or fat mixture that has not been subjected to said processing steps.

In general, the stripping processing step is preferably carried out at a pressure below 1 mbar.

In a preferred embodiment of the invention, said stripping process step is carried out at temperatures in the interval of 120-270° C., preferably at temperatures in the interval of 150-200° C.

In a preferred embodiment, the stripping processing step is at least one of short path distillation or evaporation, thin-film distillation or evaporation, falling-film distillation or evaporation and molecular distillation or any combination thereof.

As used herein the term molecular distillation is a distillation process performed at high vacuum and preferably low temperature (above 120° C.). Herein, the condensation and evaporation surfaces are within a short distance from each other, so as to cause the least damage to the oil composition. This technique is also called short-path distillation, and commercial equipment is readily available.

If at least one stripping process step is a thin-film evaporation, the process is also carried out at mixture flow rates in the range of 10-300 kg/h·m², preferably 30-150 kg/h·m², even more preferably in the range 40-150 kg/h·m².

In a further preferred embodiment of the present invention, said stripping process is a thin-film process that is carried out at 40-150 kg/h·m² or at flow rates in the range of 400-1200 kg/h at a heated thin film area of 11 m²; 36-109 kg/h·m².

In a preferred embodiment according to the invention the stripping process is carried out by a molecular distillation in the following intervals; mixture flow rates in the interval of 10-300 kg/h·m², temperatures in the interval of 120-270° C. and a pressure below 1 mbar.

In a most preferred embodiment of the invention, the molecular distillation is carried out at temperatures in the interval of 150-200° C. and at a pressure below 0.05 mbar.

In another embodiment according to the present invention, the processed oil or fat mixture is obtainable by a method comprising the following steps:
providing a mixture comprising oil or fat, said oil or fat containing environmental pollutants;
adding a volatile working fluid to the mixture, said volatile working fluid having a suitable volatility in relation to the volatility of the environmental pollutants that is to be stripped off from the fat or oil mixture;
subjecting the mixture with the added volatile working fluid to at least one stripping processing step, in which an amount of environmental pollutant present in the fat or oil is separated from the mixture together with the volatile working fluid.

An advantage of using a volatile working fluid in a process comprising at least one stripping processing step is that an amount of environmental pollutants in the mixture can more easily be stripped off together with the volatile working fluid, i.e. the environmental pollutants present in the fat or oil mixture is separated from the mixture together with the working fluid.

Preferably this is possible as long as the volatile working fluid is essentially equally or less volatile than the environmental pollutants that is to be removed from the fat or oil mixture. The stripped pollutants (components) and most of the volatile working fluid will be found in the distillate.

Another advantage of adding a volatile working fluid to an oil or fat mixture prior to a stripping process is that removal of free fatty acids is facilitated, which will result in a higher quality of the oil product.

In a preferred embodiment of the invention, the volatile environmental pollutants decreasing working fluid is a fatty acid ester (e.g. fatty acid ethyl ester or fatty acid methyl ester), a fatty acid amide or free fatty acids obtained from at least one of vegetable, microbial and animal origin, or any combination thereof. Preferably, said animal origin is fish or sea mammals, i.e. that the volatile fat or oil environmental pollutants decreasing working fluid is obtained from marine oils, e.g. from fish or from sea mammals.

In another preferred embodiment the volatile working fluid comprises at least one fatty acid ester composed of C10-C22 fatty acids and C1-C4 alcohols, or a combination of two or more fatty acid ester each composed of C10-C22 fatty acids and C1-C4 alcohols. Preferably, the volatile working fluid is at least one of amides composed of C10-C22 fatty acids and C1-C4 amines, C10-C22 free fatty acids, and hydrocarbons with a total number of carbon atoms from 10 to 40.

Most preferably, the volatile working fluid is a mixture of fatty acids from marine oils, e.g. fish body oil and/or fish liver oil, and/or ethyl or methyl esters of such marine fatty acids.

The use of a volatile working fluid comprising at least one of a fatty acid ester, a fatty acid amide, a free fatty acid and a hydrocarbon in at least one stripping process step may decrease the amount of dioxins in a fish oil with more than 95%.

In another preferred embodiment of the invention, the volatile working fluid is constituted by free fatty acids comprised in the fat or oil containing the environmental pollutants, i.e. the fat or oil itself contains free fatty acids. Here, the free fatty acids in the oil or fat acts as the volatile working fluid. Further, free fatty acids in an oil or fat also can contribute to an additive effect in a stripping process by partially acting as an internal working fluid (or by being an active part of the working fluid) in the process. Such oils or fats mentioned above could e.g. be silage oils or oils that have been stored or transported for a long period of time. This means that a volatile working fluid can be added to an oil or fat mixture prior to a stripping process and/or being comprised in the fat or oil mixture containing the environmental pollutants or toxic components.

Preferably the amount of free fatty acids in said oil or fat mixture is more than 1% by weight, more preferably more than 3% by weight, even more preferably more than 5% by weight and most preferably the non-processed mixture comprising oil or fat contains an amount of free fatty acids in the range 1-5% by weight, e.g. 2-5% by weight or 2-4% by weight.

In a preferred embodiment of the invention, the ratio of (volatile working fluid):(fat or oil is about 1:100 to 15:100. In a more preferred embodiment the ratio of (volatile working fluid):(fat or oil, being edible or for use in cosmetics) is about 3:100 to 8:100 or about 4:100 to 8:100.

Herein, the amount of environmental pollutant that is separated from the mixture is interpreted to include decreasing of an amount up to 95-99% of some environmental pollutants, i.e. a substantial removal of specific pollutants and/or toxic components from a fat or oil composition.

In one embodiment according to the present invention, the content of dioxins in the processed edible oil or fat mixture was reduced by at least 70% (w/w), more preferably by at least 80% (w/w), even more preferably by at least 90% (w/w) and most preferably by at least 95% (w/w) as compared with the edible oil or fat mixture that was not subjected to said processing steps.

In another embodiment according to the present invention, the content of dioxins and furans in the processed edible oil or fat mixture is less than 1 pg/g, more preferably less than 0.5 pg/g, even more preferably less than 0.35 pg/g and most preferably less than 0.2 pg/g.

In another embodiment, the content of brominated flame retardants in the processed edible oil or fat mixture was significantly reduced as compared with the edible oil or fat mixture that was not subjected to said processing steps. Preferably the content of brominated flame retardants in the processed edible oil or fat mixture was reduced by at least 70% (w/w), more preferably by at least 80% (w/w), even more preferably by at least 90% (w/w) and most preferably by at least 95% (w/w) as compared to the edible oil or fat mixture that was not subjected to said processing steps.

In another embodiment according to the present invention, the content of brominated flame retardants in the processed edible oil or fat mixture is less than 10 ng/g, more preferably less than 5 ng/g, even more preferably less than 3.5 ng/g and most preferably less than 2 ng/g.

In another embodiment, the content of PCB and chlorinated pesticides in the processed edible oil or fat mixture was significantly reduced as compared with the edible oil or fat mixture that was not subjected to said processing steps.

In another embodiment according to the present invention, the content of HCH in the processed edible oil or fat mixture is less than 3 ng/kg, more preferably less than 1 ng/kg, even more preferably less than 0.5 ng/kg and most preferably less than 0.3 ng/kg.

In another embodiment according to the present invention, the content of DDT in the processed edible oil or fat mixture is less than 80 ng/kg, more preferably less than 50 ng/kg, even more preferably less than 10 ng/kg, such as e.g. less than 8 ng/kg or less than 6 ng/kg, and most preferably less than 5 ng/kg.

In one embodiment according to the present invention, the content of polyaromatic hydrocarbons in the processed edible oil or fat mixture was reduced by at least 70% (w/w), more preferably by at least 80% (w/w), even more preferably by at least 90% (wlw) and most preferably by at least 95% (w/w) as compared to the edible oil or fat mixture that was not subjected to said processing steps.

In one embodiment according to the present invention, said environmental pollutants are selected from the group consisting of growth inhibiting factors, substances which accelerate sexual maturation, substances which promote oxidative damage and substances which promotes oxidation of easily oxidised components.

In another embodiment, the amount of environmental pollutant present in the fat or oil mixture that is separated from the mixture during the above mentioned processing steps is an amount that has
significant inhibitory effect on the growth of said animal; and/or
significant stimulatory effect on the tendency of an animal to enter sexual maturation; and/or
significant oxidative damage stimulatory effects in an animal; and/or
significant oxidative stress stimulatory effects in an animal; and/or
significant oxidative effect on easily oxidised components.

As previously mentioned, the stripping processing step is at least one of short path distillation or evaporation, thin-film distillation or evaporation, falling-film distillation or evaporation and molecular distillation or any combination thereof.

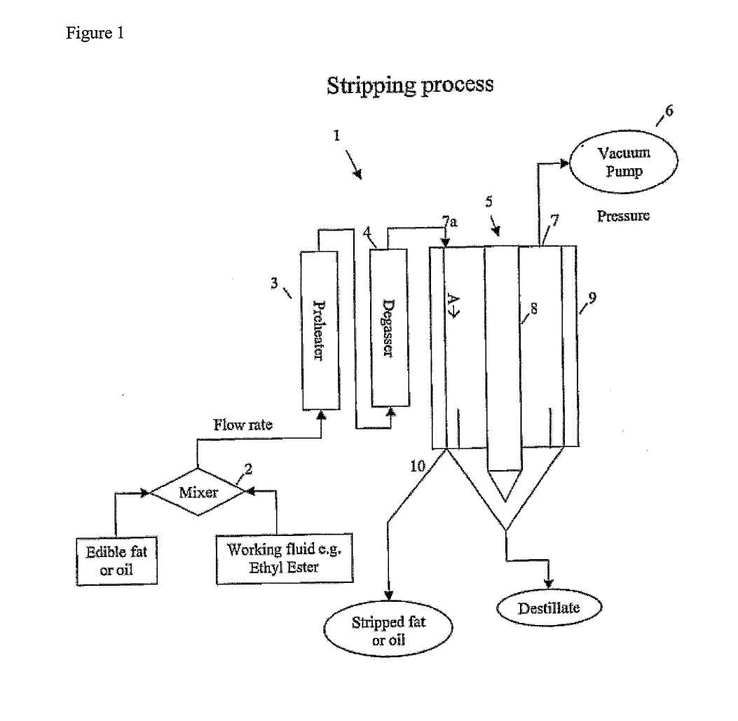

One example of a molecular distillation plant is illustrated in FIG. 1. Said molecular distillation plant (1) comprises a mixer (2), a pre-heater (3), a degasser (4), a distillation unit (5) and a vacuum pump (6). A volatile working fluid comprising an ethyl ester fraction (6% relative to the oil) may be added to a fish oil mixture and blended in a mixer (2). The oil mixture is then optionally passed through a means (3) for controlling the oil feed rate (herein about 400 kg/h), such as an ordinary throttling valve. The fish oil mixture is then preheated with a heating means (3) such as a plate heat exchanger to provide a preheated fish oil mixture. The mixture is then passed through a degassing step (4) and admitted into the molecular path distance evaporator (5), a tube (7) including the condensation (8) and evaporation (9) surface. The stripping process is carried out at a pressure between 0.1 and 0.001 mbar and at a temperature of about 200° C. The fish oil mixture to be concentrated is picked up as it enters the tube (7a) by rotating blades. The blades extend nearly to the bottom of the tube and mounted so that there is a clearance of about 1.3 mm between their tips and the inner surface of the tube. In addition, the blades are driven by an external motor. The fish oil mixture is thrown against the tube wall and is immediately spread into a thin film and is forced quickly down the evaporation surface. The film flows down by gravity and becomes concentrated as it falls. Heated walls and high vacuum strips off the volatile working fluid together with the environmental pollutants, i.e. the more volatile components (distillate) is derived to the closely positioned internal condenser (8), the less volatile components (residue) continues down the cylinder. The resulting fraction, the stripped fish oil mixture containing at least the fatty acids EPA and DHA is separated and exit through an individual discharge outlet (10).

In one preferred embodiment, the volatile working fluid with the environmental pollutants is derived to the condenser, more preferably derived to the closely positioned internal condenser.

Further, in another preferred embodiment of the invention the edible fat or oil mixture is obtained from at least one of vegetable, microbial and animal fat or oil, or any combination thereof. Preferably, the edible fat or oil mixture is a marine oil.

Marine oils that have reduced amounts of environmental pollutants are gaining popularity as well as an increasing share of the market. Consequently, removal or reduction of pollutants in e.g. fish oils of high quality as well as fish oils with inferior quality have the potential to substantially increase marketability and value. Therefore, in a more preferred embodiment of the invention the marine oil is obtained from fish or sea mammals, containing at least saturated and unsaturated fatty acids in the form of triglycerides. It is important to note that the invention is not limited to procedures were the working fluid is prepared from the same origin as the oil that is being purified.

In another embodiment, the composition according to the present invention
reduces or prevents oxidation of easily oxidised component during production and storage as compared to a composition comprising said oil or fat mixture which has not been subjected to said processing steps; and/or
increases the utilization of easily oxidised components as compared with a composition comprising said oil or fat mixture which has not been subjected to said processing steps.
reduces or prevents oxidative damage in an animal beyond what is achieved when using a composition comprising said oil or fat mixture that has not been subjected to said processing steps; and/or
reduces or prevents oxidative stress in an animal beyond what is achieved when using a composition comprising said oil or fat mixture that has not been subjected to said processing steps; and/or
promotes growth of an animal beyond what is achieved when using a composition comprising said oil or fat mixture that has not been subjected to said processing steps.

In one preferred embodiment according to the present invention, said composition is a feed composition. More preferably said feed composition is a fish feed composition. In a further embodiment according to the present invention, said composition is a health supplement. As used herein the term health supplement is interpreted to include food and food supplement to animals and/or humans, fortification of food, dietary supplement, functional (and medical) food and nutrient supplement.

In another embodiment according to the present invention, the processed oil or fat mixture amounts to at least 60% by weight of the oil or fat constituents in said composition, more preferably at least 70% by weight of the oil or fat constituents in said composition, even more preferably at lest 80% by weight of the oil or fat constituents in said composition, most preferably at least 90% by weight of the oil or fat constituents in said composition, such as e.g. 100% by weight of the oil or fat constituents in said composition.

In a further embodiment according to the present invention, the acid value of the processed oil or fat mixture is less than 2 KOH/g, preferably less than 1.5 KOH/g, more preferably less than 1 KOH/g, even more preferably less than 0.5 KOH/g such as e.g. an acid value in the range 0.4-0.2 KOH/g.

In another embodiment according to the present invention, the acid value of the processed oil or fat mixture has been reduced by at least 70%, preferably reduced by at least 80%, more preferably reduced by at least 90% and most preferably reduced by at least 95% as compared to the non-processed oil or fat mixture.

In a further embodiment, the peroxide value of the processed oil or fat mixture is less than 1.2 meq/kg, preferably less than 1.1 meq/kg, more preferably less than 1.0 meq/kg, even more preferably less than 0.9 meq/kg such as e.g. a peroxide value in the range 1.0-0.1 meq/kg or in the range 1.0-0.8 meq/kg.

In another embodiment according to the present invention, the peroxide value of the processed oil or fat mixture has been reduced by at least 15%, preferably reduced by at least 20%, more preferably reduced by at least 25% and most preferably reduced by at least 30% as compared to the non-processed oil or fat mixture.

In a further embodiment, the anisidine value of the processed oil or fat mixture is less than 17, preferably less than 16, more preferably less than 15, even more preferably less than 14.

In another embodiment according to the present invention, the anisidine value of the processed oil or fat mixture has been reduced by at least 8%, preferably reduced by at least 10%, more preferably reduced by at least 15% and most preferably reduced by at least 17% or at least 20% as compared to the non-processed oil or fat mixture.

In one preferred embodiment according to the present invention, the initial pH after slaughtering was higher in fish given the feed comprising the processed oil or fat mixture as compared with fish given the feed composition comprising the non-processed oil or fat mixture.

In a further embodiment according to the present invention, the fish fed the feed comprising the processed oil or fat mixture has firmer texture as compared with fish fed the feed comprising the non-processed oil or fat mixture. Preferably, the fish fed the feed comprising the processed oil or fat mixture has at least 1% firmer texture as compared with fish fed the feed comprising the non-processed oil or fat mixture, more preferably at least 2% firmer texture, even more preferably at least 4% firmer texture and most preferably at least 5% firmer texture.

In another embodiment, the fish fed the feed comprising the processed oil or fat mixture has 1-10% firmer texture as compared with fish fed the feed comprising the non-processed oil or fat mixture, more preferably 2-10% firmer texture, even more preferably 3-10% such as e.g. 3-5% firmer texture.

In one preferred embodiment according to the present invention, the fillet from fish fed a composition comprising the processed oil or fat mixture has a pre-rigor gaping score that is less than 0.4, preferably less than 0.3, more preferably less than 0.2, even more preferably less than 0.1 and most preferably said fish has no pre-rigor gaping.

As used herein the term environmental pollutants preferably means toxic components and/or pesticides like polychlorinated biphenyls (PCB), DDT and its metabolites, organic compounds found in the sea environment and identified as potentially harmful and/or toxic; Polychlorinated triphenyls (PCTs), dibenzo-dioxins (PCDDs), and dibenzo-furans (PCDFs), Chlorophenols and hexachlorocyclohexanes (HCHs), toxaphenes, dioxins, brominated flame retardants, polyaromatic hydrocarbons (PAH), organic tin-compounds (e.g. tributyltin, triphenyltin) and organic mercury-compounds (e.g. Methyl-Mercury).

As used herein the term oil and fat means fatty acids in at least one of the triglyceride and phospholipid forms. Generally, if the start material in the stripping process is a marine oil, the oil may be any of raw or partially treated oil from fish or other marine sources and which contains fatty acids, including polyunsaturated fatty acids, in the form of triglycerides. Typically, each triglyceride molecule in such a marine oil will contain, more or less randomly, different fatty acid ester moieties, be the saturated, monounsaturated or polyunsaturated, or long chain or short chain. Further, examples of vegetable oils or fats are corn oil, palm oil, rapeseed oil, soybean oil, sunflower oil and olive oil. Further, the fat or oil may be pre-processed in one or several steps before constituting the start material in the stripping process as described above. An example of such a pre-processing step is a deodorization process.

As used herein the term edible means edible for humans and/or animals.

As used herein the term working fluid is interpreted to include a solvent, a solvent mixture, a composition and a fraction, e.g. a fraction from a distillation process, that has a suitable volatility, comprising at least one of esters composed of C10-C22 fatty acids and C1-C4 alcohols, amides composed of C10-C22 fatty acids and C1-C4 amines, C10-C22 free fatty acids, mineral oil, hydrocarbons and bio-diesel.

As used herein the term essentially equally or less volatile is interpreted to include that the volatile working fluids having a suitable volatility in relation to the volatility of the environmental pollutants that is to be stripped off from a fat or oil mixture. Further, commonly this is the case when the volatility of the working fluid is the same or lower than the volatility of the environmental pollutants. However, the term essentially equally or less volatile is also intended to include the case when the volatile working fluid is somewhat more volatile than the environmental pollutant.

Further, as used herein the term stripping is interpreted to include a general method for removing, separating, forcing or flashing off gaseous compounds from a liquid stream.

In addition, the term “stripping processing step” preferable herein is related to a method/process for decreasing the amount of environmental pollutants in an oil or fat by one or more distilling or distillation processes, e.g. short path distillations, thin-film distillations (thin-film stripping or thin-film (steam) stripping), falling-film distillations and molecular distillations, and evaporation processes.

As used herein the term “oils with a low quality” preferably means that the oil contains high amounts of free fatty acids, that makes them less useful for nutritional purposes and that traditional alkaline refining in such oils is complicated and costly.

As used herein hydrocarbons is interpreted to include organic compounds, that are relatively large molecules composed mainly of carbon and hydrogen. They can also include nuclei of nitrogen, phosphorus, sulphur, and chlorine, among others.

As used herein bio-diesel means a commercial product (or products under development) used as an environment friendly alternative to fuel for cars comprising e.g. methyl esters from preferable vegetable or animal oils.

As used herein the term marine oils includes oil from fish, shellfish (crustaceans) and sea mammals. Non limiting examples of fish oils are e.g. Menhaden oil, Cod Liver oil, Herring oil, Capelin oil, Sardine oil, Anchovy oil and Salmon oil. The fish oils mentioned above may be recovered from fish organs, e.g. cod liver oil, as well as from the meat of the fish or from the whole fish.

As used herein the term fish feed also includes a fish larvae feed.

As used herein the term microbial oils also includes “single cell oils” and blends, or mixtures, containing unmodified microbial oils. Microbial oils and single cell oils are those oils naturally produced by microorganisms during their lifespan.

Further, an edible fat or oil mixture according to the invention can also be a blend of e.g. microbial oils, fish oils, vegetable oils, or any combination thereof.

As used herein the term free fatty acids means fatty acids in free acid form. The free fatty acids is operative as a volatile working fluid and/or included in the fat or oil.

As used herein the term “together with”, means that the volatile working fluid will be stripped off together with, combined with, or adhering the pollutants, namely that the pollutants will accompany the working fluid.

Additionally, as used herein, the term mineral oil is interpreted to include mineral oil products such as e.g. fractions from distillation processes and white spirit.

As used herein the term acid value of a fat or an oil means the amount of free acids presented in a fat or an oil equal to the number of milligrams of potassium hydroxide needed to neutralize one gram of the oil, i.e. that the term serves as an index of the efficiency of refining. This means that a high acid value is characteristic for low quality oil or fat products.

A non-limiting list of easily oxidised components are astaxanthin, cantaxanthin, tocopherols, tocotrienols, ubiquinones and lutein. Polyunsaturated fatty acids and derivatives of polyunsaturated fatty acids are also examples of easily oxidised components.

While the invention has been described in detail and with reference to specific embodiments thereof, it will be apparent for one skilled in the art that various changes and modifications, i.e. other combinations of temperatures, pressures, and flow rates during the stripping process can be made therein without departing from the spirit and scope thereof.

The invention will now be illustrated by means of the following non-limiting examples. These examples are set forth merely for illustrative purposes and summarize some results in accordance with the present invention.

### EXAMPLES

### Example 1

Feed Composition

Non-Purified Oil

Fish oil, mainly consisting of oil from herring and blue whiting.

Purified Oil

Fish oil, mainly consisting of oil from herring and blue whiting, was mixed with a working fluid (working fluid:fish oil ratio about 7:100). The working fluid used was a light ethyl ester fraction of transesterified fish oil produced as a by-product from commercial production of omega-3 concentrates (see e.g. example 1 and 4 in WO2004007654). The obtained mixture was then subjected to molecular distillation at a rate of 350 l/h and a temperature of 247° C. The obtained product was then cooled to a temperature of about 30° C.

Experimental Diets

The experimental diets were processed using a Wenger (TX 52) co-rotating twin screw pilot-scale extruder at fixed and defined conditions (less than one minute processing time and a max temperature of 130° C.). Fat content in mash was 12% on dry matter basis. The extruded pellets were dried in a Paul Klöckner (Type 200.2) carousel dryer and the rest of the fish oil was added in a Dinnissen vacuum coater. The ingredients are as set forth in table 1-3.

Two sets of different feed compositions were produced. In set 1, said fish oil is the above mentioned purified oil. In set 2, said fish oil is the above mentioned non-purified oil.

TABLE 1
Feed recipe (kg) for pellet 4 mm diameter. Fiskeriforskning
(now Nofima Ingredients) batch numbers in parentheses.

| | |
|---|---|
| Fish meal (268/06; | 84.62 |
| Norwegian, mostly | |
| herring) | |
| Soy protein | 11.25 |
| concentrate (155/05) | |
| Wheat gluten | 9.38 |
| (156/05) | |
| Soy meal, extracted | 11.25 |
| (252/06) | |
| Fish oil¹(Norwegian) | 43.69 |
| Wheat meal (209/06) | 22.69 |
| Vitamin mix² | 3.75 |
| Mineral mix³ | 0.75 |
| Carophyll Pink | 0.113 |
| (10%) | |
| Yttrium oxide⁴ | 0.019 |
| Sum | 187.5 |

TABLE 2
Feed recipe (kg) for pellet 6 mm diameter.
Fiskeriforskning (now Nofima Ingredients)
batch numbers in parentheses.

| | |
|---|---|
| Fish meal (268/06; | 352.9 |
| Norwegian, mostly | |
| herring) | |
| Soy protein | 41.0 |
| concentrate (155/05) | |
| Wheat gluten (156/05) | 32.8 |
| Soy meal, extracted | 49.2 |
| (252/06) | |
| Fish oil¹ (Norwegian) | 209.1 |
| Wheat meal (209/06) | 114.8 |
| Vitamin mix² | 16.4 |
| Mineral mix³ | 3.28 |
| Carophyll Pink (10%) | 0.492 |
| Yttrium oxide⁴ | 0.082 |
| Sum | 820.0 |

TABLE 3
Feed recipe (kg) for pellet 9 mm diameter.
Fiskeriforskning (now Nofima Ingredients)
batch numbers in parentheses.

| | |
|---|---|
| Fish meal (214/07) | 423.8 |
| Norwegian, mostly | |
| herring) | |
| Fish meal (217/07, | 130.2 |
| Peruvian, anchova) | |
| Soy protein | 124.0 |
| concentrate (181/07) | |
| Wheat gluten (bl.10-07) | 62.0 |
| Soy meal, extracted | 93.0 |
| (180/07) | |
| Fish oil¹ (Norwegian) | 437.1 |
| Wheat meal (174/07) | 241.8 |
| Vitamin mix² | 31.0 |
| Mineral mix³ | 6.2 |
| Carophyll Pink (10%) | 0.930 |
| Sum | 1550 |

¹The fish oil consisted mainly of oil from herring and blue whiting.
²The vitamin mix provided per kg feed: vitamin D₃, 3000 I.U., 160 mg; vitamin E (Rovimix, 50%), 160 mg; thiamine, 20 mg; riboflavin, 30 mg; pyridoxine-HCl, 25 mg; vitamin C (Rovimix Stay C, 35%), 200 mg; calcium pantothene, 60 mg; biotin, 1 mg; folic acid, 10 mg; niacin, 200 mg; vitamin B12, 0.05 mg; menadion bisulphate, 20 mg.
³The mineral mix provided per kg feed: magnesium, 500 mg; potassium, 400 mg; zinc, 80 mg; iron, 50 mg; manganese, 10 mg; copper, 5 mg.
⁴Yttrium oxide was added for feed absorption measurements.

The person skilled in the art will realise that the feed composition could be varied in a number of ways, for example by substituting part of the fish oils by vegetable oil.

### Example 2A

Reduced Loss of Astaxanthin in Feed During Production and Storage

Two sets of different feed compositions were produced as described in example 1. For all feed compositions the commercial pigment Carophyll Pink (DSM) was added in identical concentrations during the vacuum coating process. The aim was to add 60 mg/g astaxanthin to the feed. The results are shown in Table 4.

TABLE 4
Astaxanthin conecentrations (mg/kg) in feed pellets. The feed was
produced at Fiskeriforskning (now Nofima), Bergen, Norway, and
the analysis of astaxanthin was performed at Akvaforsk (Now
Nofima), Sunndalsora, Norway.

| | Pellet size (mm) | | | | |
|---|---|---|---|---|---|
| | 4 | 4 | 6 | 9 (batch 1) | 9 (batch 2) |
| Approx. | 2 | 11 | 2 | 2 | 1 |
| storage time | | | | | |
| before | | | | | |
| analysis | | | | | |
| (months) | | | | | |
| Feed with | 61.4 | 49.5 (81% of | 61.4 | 58.2 | 60.5 |
| purified oil | | level at 2 | | | |
| | | months) | | | |
| Feed with the | 56.4 | 42.4 (75% of | 58.8 | 56.2 | 57.2 |
| same oil | | level at 2 | | | |
| without | | monts) | | | |
| purification | | | | | |

### Example 2B

Better Utilisation of the Astaxanthin Pigment

Fanned Atlantic salmon was fed with feed as described in example 2A (group W=control; group E=fish having received feed with processed oil). After slaughtering, bulk weighing gave an average weight of 4618 g for all the fish in Group W and 4929 g for all the fish in Group E.

Visual colour of salmon fillet is commonly measured as Colour Score by comparison with the so-called SalmoFan™ from the company DSM. It is desirable to obtain high values. The Table below gives SalmoFan™ Colour Score at front, dorsal fin, tail and belly from fillet of Atlantic salmon fed either control diet (W) or diet with processed fish oil cleaned for persistent environmental pollutants (E). The values are average values from 10 randomly sampled fish from each net pen, i.e. 40 fish from each group.

The so-called Norwegian Quality Cut (NQC) is a standardised muscle section of the fish (Norwegian Standard method NS 9401, 1994). The Table also includes average NQC SalmoFan™ Colour score for 5 randomly sampled fish from each net pen, i.e. 20 fish from each group. The sample fish for NQC were different from the sample fish for fillet measurements.

TABLE 6
Average NQC SalmoFan ™ Colour score

| | W | E |
|---|---|---|
| Front | 29.9 | 30.5 |
| Dorsal fin | 29.5 | 30.0 |
| Tail | 29.0 | 29.6 |
| Belly | 27.4 | 28.3 |
| NQC | 29.9 | 30.6 |

The Table documents that on average the fish in the E group had a deeper red, and thus more desirable, colour than the fish in the W (control) group.

The individual values for the NQC measurements are given below:

TABLE 7
Individual values for the NQC measurements

SalmoFan ™ Colour Score

| Net Pen | Individual values | | | | | Average |
|---|---|---|---|---|---|---|
| 701 (W) | 29 | 28 | 29 | 30 | 28 | 28.8 |
| 702 (W) | 30 | 31 | 30 | 31 | 29 | 30.2 |
| 705 (W) | 30 | 30 | 31 | 30 | 30 | 30.2 |
| 706 (W) | 31 | 31 | 30 | 29 | 30 | 30.2 |
| 703 (E) | 30 | 31 | 31 | 30 | 30 | 30.4 |
| 704 (E) | 31 | 31 | 31 | 30 | 30 | 30.6 |
| 707 (E) | 31 | 31 | 31 | 30 | 31 | 30.8 |
| 708 E | 31 | 30 | 30 | 31 | 31 | 30.6 |

As expected there are overlaps of individual values between the two groups. However, there are no overlaps between the average values from net pens from the two groups of fish.

The feeds were prepared with addition of identical concentrations of astaxanthin. As the study documented a better feed conversion for the fish having received a diet with purified oil (Group E) compared to the control (Group W), this means that there had been given less feed per kg slaughtered salmon of the E group compared to the control group (W). This also means that there had been less astaxanthin cost per kg salmon in the E group compared to the W group. In contrast to this, the salmon in the E group surprisingly showed a deeper and more desirable colouration than the salmon in the control group.

Very surprisingly it has thus been showed that purification of the feed oil leads to better utilisation of the expensive astaxanthin pigment that is added to the feed.

### Example 2C

Preventing Oxidation of Easily Oxidised Components

A crude fish oil was purified according to the process of the present invention. The short path distillation was carried out at 180° C. The oil had an acid value 8.9 KOH/gram, i.e. a free fatty acid content of approx 4.5%. 4.0% of a distilled fatty acid ethyl ester from fish oil was added to the oil as working fluid in addition to the free fatty acid internal working fluid. Before purification the oil had a peroxide value of 1.3 meq/kg and an anisidine value of 17.0.

After purification the acid value was 0.3 KOH/gram, the peroxide value was 0.9 meq/kg and the anisidine value was 14.0.

Before purification the oil contained approx. 240 mg/kg of d-alpha-tocopherol. As expected, after purification the content of d-alpha-tocopherol was somewhat reduced.

Astaxanthin (8 mg/kg) was added to both the starting oil and to the oil processed according to the present invention. 3 grams portions of both oils were stored in normal atmosphere at 30° C. in petri dishes with a diameter of 60 mm. After 15 days the processed oil was substantially free of astaxanthin, i.e. substantially all the astaxanthin had oxidised, while the untreated fish oil contained approx. 6 mg/g astaxanthin. This shows that astaxanthin oxidises more rapidly in the treated oil, which can be explained by this oil containing less tocopherol than the non-treated-oil.

Based on this observation it is even more surprising that astaxanthin is more stable during production and storage in feed that is produced with oil processed according to the present invention than in feed that contains the same oil, but without purification (See Example 2A). Possibly the reduction of tocopherol in the treated oil had somehow been compensated by the other components in the feed raw materials (which were identical for both feeds). However, this remains a speculation, and the inventors do not limit the stabilisation effect on astaxanthin to any specific explanation.

As in the experiment above, d-alpha-tocopherol (approx. 150 mg/kg) was added both to the untreated oil and to the oil processed according to the present invention. After addition analysis showed that the untreated oil contained 387 mg/kg of d-alpha-tocopherol, while the processed oil contained 352 mg/kg of d-alpha-tocopherol. 3 grams portions of both oils were stored in normal atmosphere at 30° C. in petri dishes with a diameter of 60 mm.

The content of d-alpha-tocopherol was analyzed in both oils at intervals as shown in table 12.

TABLE 12
Concentration of d-alpha-tocopherol in mg/kg and percent of
concentration at day 0 for 3 grams samples of oils stored in
petri dishes (i.d. 60 mm) at 30° C.

| | Storage time (days) | | | | |
|---|---|---|---|---|---|
| Oil | 0 | 9 | 11 | 14 | 25 |
| Untreated | 387 (100) | 250 (65) | 222 (63) | 130 (34) | 0 |
| Purified | 352 (100) | 270 (78) | 243 (69) | 130 (37) | 0 |

Very surprisingly, d-alpha-tocopherol was more stable in the purified oil (processed oil) compared to the untreated oil. This is in contrast to the results with astaxanthin with the same oil (see above), and shows that treatment of an oil according to the present invention surprisingly protects tocopherol against oxidation.

### Example 3

Growth, Health Effects and Oxidative Damage

Farmed Atlantic salmon was fed with feed as described in Table 4. At approx. 900 and approx 1600 grams size, fish from both groups were analysed with regards to certain biological parameters. The results are shown in Table 5.

TABLE 5
Growth and biological analyses of salmon fed with to identical diets, except that in
one of the diets the oil had been treated in order to remove undesirable elements.

| | Sampling 1 | | Sampling 2 | |
|---|---|---|---|---|
| | Non-purified oil | Purified oil | Non-purified oil | Purified oil |
| Weight of fish | 0.902 ± 0.04 | 0.901 ± 0.05 | 1.547 ± 0.061 | 1.654 ± 0.041 |
| (kg) (average 4 | | | | |
| net pens per group) | | | | |
| FCR (feed | 0.83 ± 0.01 | 0.83 ± 0.01 | 0.88 ± 0.02¹ | 0.80 ± 0.01¹ |
| conversion ratio) | | | | |
| Liver EROD | 6.4 ± 0.79 | 3.8 ± 0.44 | 13.16 ± 1.2 | 8.39 ± 0.7 |
| Adipose Cyt C | 1.10 ± 0.01 | 1.00 ± 0.11 | 2.82 ± 0.5 | 1.00 ± 0.3 |
| oxidase | | | | |
| Adipose SOD | 3.79 ± 0.6 | 3.61 ± 0.5 | 3.87 ± 1.1 | 1.92 ± 0.1 |

¹Average between first and second sampling
FCR: feed conversion ratio, i.e. relative ratio of feed to growth
EROD: 7-Ethoxyresorufib-O-deethylase activity
Cyt C oxidase: Cytochrome C oxidase activity
SOD: Superoxide dismutase activity

Histological evaluation of kidney, liver and intestine displayed normal organ morphology and structure of tissues and cells in both dietary groups. In agreement with the histological evaluation, blood biochemistry markers which may indicate injury to, or altered function of the liver showed no difference between the two groups.

In aquaculture it is preferable that FCR is as low possible, in order to ensure a high growth from a given amount of feed. A FCR of 0.80 means that 0.80 kg of feed results in a weight gain of 1.00 kg. Surprisingly, the feed containing the purified oil was found to have significant lower FCR as compared to feed containing the non-purified oil.

The cytochrome P450 (EROD) activity is often applied to investigate if exposure to contaminants has resulted in biochemical and physiological responses. In the example EROD analyses showed lower, more desirable values for the fish that has been fed with purified oil.

A high value for Cyt C oxidase, measured as the ratio in the presence and in the absence of the detergent n-nondecyl β-D-maltoside, is used as a relative measurement of peroxidation damage of mitochondria.

Reactive oxygen species (ROS) (free radicals, reactive anions containing oxygen, molecules containing oxygen atoms that can produce free radicals or are activated by free radicals) induce cell damage. A high SOD indicate ROS-induced damage to cells.

### Example 4

Reduced Tendency of an Animal to Enter Sexual Maturation

The study described in Example 3 was conducted under standard procedures for light treatment in order to reduce the possibility of sexual maturation. However, at a weight of around 3 kg some of the fish described in Example 3 started showing signs of sexual maturation. During bulk-weighing fish that visually showed signs of maturation were removed, and for these fish the gonadosomatic index (GSI) (relative weight of gonads compared to the complete body weight) was determined. For the diet with non-purified oil, 17.8% of the fish had reached maturation, with an average GSI of 7.5%, while for the diet with purified oil 13.0% of the fish had reached maturation, with an average GSI of 7.0%.

### Example 5

Fatty Acid Composition

Two different feed oils were analysed for eicosapentaenoic acid (EPA) and docosahexaenoic (DHA) content before (W) and after (E) being processed as disclosed in example 1.

TABLE 8
Content of EPA and DHA in feed oil

| | |
|---|---|
| Oil 1 | Oil 2 |

| | (W) | (E) | (W) | (E) |
|---|---|---|---|---|
| EPA | 5.5 | 5.4 | 6.3 | 6.1 |
| DHA | 10.4 | 10.2 | 11.3 | 10.8 |

Thus, treatment according to the present invention may lead to a small reduction in content of EPA and DHA.

Fillets from the same fish that were analysed for colour in Example 2B were homogenised and analysed for fatty acid composition. The fish had received feed produced from both oils that are described in table 8 above (identical relative amounts for both fish groups).

Analysis of the fish fillets gave results as shown in table 9 below.

TABLE 9
Content of EPA and DHA in fish fillets

| | |
|---|---|
| Fatty acid (% | Dietary group |

| relative content) | W | E |
|---|---|---|
| Sum saturated | 21.6 ± 0.08 | 21.9 ± 0.11 |
| Sum monounsaturated | 55.0 ± 0.09 | 54.2 ± 0.15* |
| Sum polyunsaturated | 20.9 ± 0.08 | 21.5 ± 0.14* |
| Sum omega-6 | 3.9 ± 0.05 | 3.9 ± 0.01 |
| Sum omega-3 | 15.0 ± 0.08 | 15.7 ± 0.08* |
| EPA | 4.5 ± 0.03 | 4.8 ± 0.03* |
| DHA | 7.7 ± 0.05 | 7.9 ± 0.06* |

*Significant differences (p < 0.05)

Thus, the fish that had received feed with purified oil surprisingly had fillets with higher content of EPA, DHA and total omega-3 fatty acids than fillets of fish that had received the same feed oil, but without purification. This is even more surprising as the purified feed oils tended to contain less EPA and DHA than the oil before purification (table 8).

### Example 6

Hepatosomatic Index

Enlarged liver size may be a sign of illness or reduced health, and is undesirable in farmed fish.

For the same fish that were analysed for colour in Example 2B, the liver weights were recorded. The hepatosomatic index is defined as the relative weight of the liver (%) compared to the weight of the whole fish. The hepatosomatic index for the fish in Group W was 1.18%, while the hepatosomatic index for the fish in Group E was only 1.11% (p-value 0.008).

It is highly surprising that purification of the feed oil can give significant reduction of the hepatosomatic index, and illustrates the positive health effect of purifying the feed oil.

For some farmed fish species, i.e. cod, pollock, saithe and coalfish enlarged liver size compared to wild fish is a significant economical problem, as much of the feed is just utilised for increasing the liver size. Typically, the hepatosomatic index for farmed cod can be above 10%. Thus, purification of feed oil according to the present invention will be of value for improving the economy of the fish farming industry by reducing the amount of feed that is utilised for producing enlarged fish livers.

### Example 7A

Fillet Quality “Consumer Test”

A consumer test revealed no sensory difference between fish given a feed comprising the processed oil or fat mixture and fish given a feed comprising the non-processed oil or fat mixture. This shows that purification of the feed oil does not negatively affect the taste or other sensory characteristics of the fish.

### Example 7B

Fillet Quality “pH, Texture, Colouration and Gaping of Fillet”

After slaughtering muscle pH was measured. The measurements were performed in the area anterior to the dorsal fin, above the lateral line. The measurements showed that the initial pH was higher in salmon fed the diet comprising the processed oil or fat mixture (diet E) compared with salmon fed the diet comprising the non-processed oil or fat mixture (diet W). The average for 20 individuals from each treatment (5 randomly sampled salmon per net-pen) was pH 7.3 for Diet E and 7.1 for Diet W. There was still a difference after 8 hours storage, while after 24 hours the pH was similar for both groups (pH 6.7). It is known by the person skilled in the art that lowered initial pH coincides with reduced shelf life.

It was found that salmon fed Diet E had 3-5% firmer texture compared with Diet W. Similar results were obtained for fillets analysed after 4, 5 and 11 days of ice storage and after 4 months storage at −20° C. (average for 20 individuals from each treatment). Salmon with texture strength below 6 Newton is considered too soft for applications like smoking (Mørkøre, T., Rørå, A. M. B., 1999. Hvordan måle tekstur. Norsk Fiskeoppdrett Tema, 38-39.) After frozen storage for 4 months the discard rate based on this criterion for fish from Diet E was only half of the discard rate for fish from Diet W. Also, the colouration of fish from Diet E was better than fish from Diet W after frozen storage (0.5 units based on the SalmoFan Scale).

Studies have shown a significant relationship between flesh softening and accelerated growth rate for fanned fish (Roth B., Johansen S. J. S., Suontama J., Kiessling A., Leknes O., Guldberg B. & Handeland S. (2005) Seasonal variation in flesh quality, comparison between large and small Atlantic salmon (Salmo salar) transferred into seawater as 0+ or 1+ smolts. Aquaculture 250, 830-840. Folkestad, A., Rørvik, K-A., Kolstad, K., Mørkøre, T. (2008) Growth rates of individual farmed Atlantic salmon Salmo salar L. influence the texture of raw and smoked fillets. Aquaculture Research 39, 329-332.)

In the present study the fish fed Diet E had a higher growth compared with Diet W. Very surprisingly the higher growth rate of the salmon fed Diet E was not reflected in a poorer texture quality; i.e. that Diet E gave improved production efficiency without affecting the muscle structure negatively. On the contrary, fish fed Diet E had improved texture compared to Diet W.

The degree of fillet gaping was evaluated after rough handling of the fillet according to a method by Andersen et al. (Andersen, U. S., Strømsnes, A. N., Steinsholt, K., Thomassen, M. S., 1994. Fillet gaping in fanned Atlantic salmon (Salmo salar). Norw. J. Agric. Sci. 8, 165-179). Gaping is observed as slits or holes in the fillet surface. The gaping score goes from 0=no gaping, to 5=extreme gaping. This test is now considered part of an “Industry test” describing general quality of salmon and suitability to secondary processing. The fish flesh is very elastic before the muscle enters rigor mortis. Therefore, the gaping score is very low before rigor development, and no difference was expected between the groups. However, while no pre rigor gaping was observed from fish from Diet E, a gaping score of 0.5 was observed for fish from Diet W. After vacuum packing of fillets after rigor resolution and 5 days storage on ice the gaping score for fish from Diet E was still 0, while the average score for fish from Diet W was 0.2.

### Example 8

Environmental Pollutants

The contents of cholesterol, vitamin E, vitamin A, vitamin D and PCBs and dioxins in fish oil 1 (based on 17% Blue whiting, 49% herring, 17% capelin and 17% by-products from herring) before (W) and after (E) removal of environmental pollutants by short path distillation is shown in table 10.

TABLE 10
Contents of certain ingredients in fish oil before and after purification

| | Fish oil 1 | | |
|---|---|---|---|
| | | | % |
| | W | E | reduction |
| Total cholesterol, mg/g | 10.3 | 3.8 | 63.1% |
| Tocoferol, mg/g | 0.16 | 0.04 | 75.0% |
| Vitamin A, μg/g | 95.1 | 74.2 | 22.0% |
| Vitamin D, μg/g | 1.62 | 0.77 | 52.5% |
| 7 PCB¹, ng/g | 68.60 | 0.50 | 99.3% |
| Total PCB², ng/g | 173.00 | 1.07 | 99.4% |
| Dioxins and Furans³ (TE WHO), pg/g | 5.03 | 0.18 | 96.4% |
| Mono-ortho PCB⁴ (TE WHO), pg/g | 1.97 | 0.03 | 98.5% |
| Non-ortho PCB⁵ (TE WHO), pg/g | 5.09 | 0.04 | 99.4% |
| Dioxin-like PCB⁶, pg/g | 7.06 | 0.07 | 99.0% |

¹7 PCB = sum PCB(28 + 52 + 101 + 118 + 138 + 153 + 180)
²Sum PCB = sum of observed PCB (nono- and di-CB are not included)
³Sum Polychlorinated dibenzo-para dioxins (dioxins) + Polychlorinated dibenzofurans (furans)
⁴Mono-otho PCB = PCB(105 + 114 + 118 + 123 + 156 + 157 + 167 + 189)
⁵Nonortho PCB = PCB(77 + 81 + 126 + 169)
⁶Mono-ortho PCB + nonortho PCB

The contents of hexachlorocyclohexane (HCH) and dichlorodiphenyltrichloroethane (DDT) (ng/kg) in fish oil 1 before (W) and after (E) removal of environmental pollutants by short path distillation is shown in table 11.

TABLE 11
Contents of HCH and DDT in fish oil before and after purification

| | W | E | % reduction |
|---|---|---|---|
| α-HCH, ng/kg | 2.72 | 0.1¹ | >96.32% |
| β-HCH, ng/kg | 1.19 | 0.06^{1,2} | >94.96% |
| γ-HCH, ng/kg | 0.93 | 0.09¹ | >90.32% |
| SUM HCH | 4.84 | 0.25 | >94.83% |
| o,p′-DDE | 0.81 | 0.03 | 96.30% |
| p,p′-DDE | 56.7 | 4.17 | 92.65% |
| o,p′-DDD | 2.27 | 0.04^{1,2} | >98.24% |
| p,p′-DDD | 18.9 | 0.37¹ | >98.04% |
| o,p′-DDT | 3.13¹ | 0.2 | 93.61% |
| p,p′-DDT | 9.98 | 0.09 | 99.10% |
| SUMDDT | 91.8 | 4.9 | >94.66% |

¹Lower than 10 times method blank
²Isotope ratio deviates more than 20% from the theoretical value

The contents of polybrominated diphenyl ether (ng/g) in fish oil 1 before (W) and after (E) removal of environmental pollutants by short path distillation is shown in table 13.

TABLE 13
Content of poly brominated diphenyl ether in fish oil 1 before and after
purification

| Compound (structure) | IUPAC-no | W | E | % reduction |
|---|---|---|---|---|
| 4,4′-DiBB | 15 | 0.04² | 0.04 | |
| 2,2′,4,4′,5,5′-HexBB | 153 | 0.03 | 0.01¹ | >66.7% |
| 2,4,4′-TriBDE | 28 | 0.4 | 0.01¹ | >97.5% |
| 2,2′,4,4′-TetBDE | 47 | 4.67 | 0.29² | 93.8% |
| 2,3′,4,4′-TetBDE | 66 | 0.28² | 0.02¹ | |
| 2,2′,4,5 + 2,3′,4′,6-TetBDE | 49 + 71 | 1.76 | 0.03 | 98.3% |
| 3,3′,4,4′-TetBDE | 77 | 0.01¹ | 0.01¹ | |
| 2,2′,3,4,4′-PenBDE | 85 | 0.01¹ | 0.02¹ | |
| 2,2′,4,4′,5-PenBDE | 99 | 0.77 | 0.22² | 71.4% |
| 2,2′,4,4′,6-PenBDE | 100 | 0.7 | 0.07² | 90.0% |
| 2,3′,4,4′,6-PenBDE | 119 | 0.16 | 0.01¹ | >93.8% |
| 2,2′,3,4,4′,5′-HexBDE | 138 | 0.04¹ | 0.03¹ | |
| 2,2′,4,4′,5,5′-HexBDE | 153 | 0.2 | 0.08 | 60.0% |
| 2,2′,4,4′,5,6′-HexBDE | 154 | 0.54 | 0.06 | 88.9% |
| 2,2′,3,4,4′,5′,6-HepBDE | 183 | 0.03 | 0.03² | |
| 2,2′,3,3′,4,4′,5,6′-OctBDE | 196 | 0.08¹ | 0.09¹ | |
| 2,2′,3,3′,4,4′,5,5′,6-NonBDE | 206 | 0.12¹ | 0.12¹ | |
| SUM PBDE | | 13.87 | 1.92 | >85.2% |
| Most common PBDEs³ | | 7.28 | 0.73 | >90.0% |
| Most common PBDEs in fish | | 8.84 | 0.68 | >92.3% |

¹Below detection level
²Isotope ratio deviates more than 20% from theoretical value. This may be due to instrumental noise oriand chemical interference.
³PBDE (28, 47, 99, 100, 153 and 154). Most common congeners found in animals (NIFES's Seafood data (http://www.nifes.co/ [symbol] data/index.php? [symbol] _id=&laps_id=2)).
⁴PEDE (28, 47, 49, 71, 99, 100, 154). Most common PDBEs found in fish. [symbol]
indicates data missing or illegible when filed

Content of environmental pollutants in fillet of atlantic salmon fed either a feed comprising non-processed fish oil (W) or a feed comprising fish oil which has been processed in order to remove environmental pollutants (E) is shown in table 14.

TABLE 14
Content of environmental pollutants in fish fillet before and
after purification

| | Diet | | |
|---|---|---|---|
| Variable | W | E | p-value |
| 7 PCB¹, ng/g | 18.98 | 6.75 | 0.0005 |
| Total PCB², ng/g | 47.40 | 16.83 | 0.0004 |
| Mono-ortho PCB³, ng/g | 0.50 | 0.16 | 0.0004 |
| PCDD⁴, pg/g, TE(WHO) | 0.12 | 0.13 | 0.9085 |
| PCDF⁵, pg/g, TE(WHO) | 0.54 | 0.08 | <0.0001 |
| Nonortho PCB⁶, pg/g, TE(WHO) | 1.07 | 0.16 | <0.0001 |
| PCDD + PCDF⁷, pg/g, TE(WHO) | 0.66 | 0.20 | 0.0006 |
| Dioxin like PCB⁸, pg/g, TE(WHO) | 1.57 | 0.32 | <0.0001 |
| Dioxins + dioxin like PCB⁹, pg/g, TE (WHO) | 2.23 | 0.52 | <0.0001 |

¹7 PCB = sum PCB(28 + 52 + 101 + 118 + 138 + 153 + 180)
²Sum PCB = sum of observed PCB (nano- and di-CB are not included)
³Mono-ortho PCB = PCB(105 + 114 + 118 + 123 + 156 + 157 + 167 + 189)
⁴Polychlorinated dibenzo-para dioxins
⁵Polychlorinated dibenzofurans
⁶Nonortho PCB = PCB(77 + 81 + 126 + 169)
⁷Dioxins-generic term
⁸Mono-ortho PCB + nonortho PCB
⁹PCDD + PCDF + mono-ortho PCB + nonortho PCB

Content of poly brominated diphenyl ether in fillet of atlantic salmon fed either a feed comprising non-processed fish oil (W) or a feed comprising fish oil which has been processed in order to remove environmental pollutants (E) is shown in table 15.

TABLE 15
Content of poly brominated diphenyl ether in fish fillet before and after
purification

| Compound (structure) | IUPAC-no | W | E | p-value |
|---|---|---|---|---|
| 4,4′-DiBB | 15 | | | |
| 2,2′,4,4′,5,5′-HexBB | 153 | | | |
| 2,4,4′-TriBDE | 28 | 0.08 | 0.01² | <0.0001 |
| 2,2′,4,4′-TetBDE | 47 | 1.18 | 0.15 | <0.0001 |
| 2,3′,4,4′-TetBDE | 66 | 0.05 | 0.01² | <0.0001 |
| 2,2′,4,5′ + 2,3′,4′,6-TetBDE | 49 + 71 | 0.30 | 0.04 | <0.0001 |
| 3,3′,4,4′-TetBDE¹ | 77 | — | — | — |
| 2,2′,3,4,4′-PenBDE¹ | 85 | — | — | — |
| 2,2′,4,4′,5-PenBDE | 99 | 0.17 | 0.04 | 0.0001 |
| 2,2′,4,4′,6-PenBDE | 100 | 0.18 | 0.03 | <0.0001 |
| 2,3′,4,4′,6-PenBDE | 119 | 0.02 | 0.01² | 0.0025 |
| 2,2′,3,4,4′,5′-HexBDE¹ | 138 | — | — | — |
| 2,2′,4,4′,5,5′-HexBDE | 153 | 0.04 | 0.02 | 0.0036 |
| 2,2′,4,4′,5,6′-HexBDE | 154 | 0.14 | 0.05 | <0.0001 |
| 2,2′,3,4,4′,5′,6-HepBDE | 183 | — | — | — |
| 2,2′,3,3′,4,4′,5,6′-OctBDE | 196 | — | — | — |
| 2,2′,3,3′,4,4′,5,5′,6-NonBDE | 206 | — | — | — |
| SUM PBDE³ | | 3.61 | 1.63 | 0.0004 |
| Most common PBDE⁴ | | 1.80 | 0.30 | <0.0001 |
| Most common PBDEs in fish | | 2.06 | 0.32 | <0.0001 |

¹Below detection level for Control diet group (W)
²Below detection limit for diet E, but [symbol] for diet W
³SUM PBDE includes results below detection limit
⁴PBDE (28, 47, 99, 100, 153 and 154). Most common congeners found in animals
⁵PBDE (28, 47, 49, 71, 99, 100, and 154). [symbol]
indicates data missing or illegible when filed

EU Directive 199/2006 EC gives maximum sum of dioxins and furans (WHO-PCDD/F-TEQ) in muscle meat of fish and fishery products of 4.0 pg/g, and maximum sum of dioxins, furans and dioxin-like PCBs (WHO-PCDD/F-PCB-TEQ) of 8.0 pg/g.

Table 14 shows that in the control fish for this study these values were 0.66 pg/g and 2.23 pg/g respectively, i.e. less than one third of the upper EU limits. Based on these relatively low concentrations of dioxins and dioxin-like contaminants the observed growth, health and quality effects by purification of the feed are highly surprising.

Table 15 shows that levels of brominated flame retardants were reduced by more than 80% in the fillet of fish that received purified oil in the diet. It is possible that this reduction, and reduction in other contaminants, also influenced the improved growth and health in the fish receiving purified feed. However, other unknowns effects of the purification may also have caused the beneficial growth, health and quality effects.

1. Composition comprising an edible processed oil or fat mixture, said processed oil or fat mixture being obtainable by a method comprising the following steps:
providing a mixture comprising oil or fat, said oil or fat containing environmental pollutants;
subjecting the mixture to at least one stripping processing step, in which an amount of environmental pollutant present in the mixture is separated from the mixture.

2. Composition according to claim 1, comprising an edible processed oil or fat mixture for reducing the tendency of an animal to enter sexual maturation, said processed oil or fat mixture being obtainable by a method comprising the following steps:
providing a mixture comprising oil or fat, said oil or fat containing environmental pollutants;
subjecting the mixture to at least one stripping processing step, in which an amount of environmental pollutant present in the mixture is separated from the mixture.

3. Composition according to claim 1, comprising an edible processed oil or fat mixture for reducing or preventing oxidative damage and/or oxidative stress in an animal, said processed oil or fat mixture being obtainable by a method comprising the following steps:
providing a mixture comprising oil or fat, said oil or fat containing environmental pollutants;
subjecting the mixture to at least one stripping processing step, in which an amount of environmental pollutant present in the mixture is separated from the mixture.

4. Composition according to claim 1, comprising an edible processed oil or fat mixture and at least one easily oxidised component, said processed oil or fat mixture being obtainable by a method comprising the following steps:
providing a mixture comprising oil or fat, said oil or fat containing environmental pollutants;
subjecting the mixture to at least one stripping processing step, in which an amount of environmental pollutant present in the mixture is separated from the mixture.

5. Composition according to claim 1, comprising an edible processed oil or fat mixture, said processed oil or fat mixture being obtainable by a method comprising the following steps:
providing a mixture comprising oil or fat, said oil or fat containing environmental pollutants;
subjecting the mixture to at least one stripping processing step, in which an amount of environmental pollutant present in the mixture is separated from the mixture;
for promoting growth of a non-human animal beyond what is achieved when using a composition comprising said oil or fat mixture that has not been subjected to said processing steps.

6. Composition according to claim 1, comprising an edible processed oil or fat mixture, said processed oil or fat mixture being obtainable by a method comprising the following steps:
providing a mixture comprising oil or fat, said oil or fat containing environmental pollutants;
subjecting the mixture to at least one stripping processing step, in which an amount of environmental pollutant present in the mixture is separated from the mixture;
for avoiding enlarged liver size in fish.

7. Feed composition comprising an edible processed oil or fat mixture, said processed oil or fat mixture being obtainable by a method comprising the following steps:
providing a mixture comprising oil or fat, said oil or fat containing environmental pollutants;
subjecting the mixture to at least one stripping processing step, in which an amount of environmental pollutant present in the mixture is separated from the mixture.

8. Composition according to claim 1, wherein said at least one stripping processing step is selected from short path distillation or evaporation, thin-film distillation or evaporation, falling-film distillation or evaporation and molecular distillation.

9. Composition according to claim 8, wherein said one thin-film evaporation process is carried out at a mixture flow rate in the range 30-150 kg/h·m².

10. Composition according to claim 1, wherein said stripping processing step is carried out at temperatures in the interval of 120-270° C.

11. Composition according to claim 1, wherein said stripping processing step is carried out at a pressure below 1 mbar.

12. Composition according to claim 1, said method comprising the following steps:
providing a mixture comprising oil or fat, said oil or fat containing environmental pollutants;
adding a volatile working fluid to the mixture, said volatile working fluid having a suitable volatility in relation to the volatility of the environmental pollutants that is to be stripped off from the fat or oil mixture;
subjecting the mixture with the added volatile working fluid to at least one stripping processing step, in which an amount of environmental pollutant present in the fat or oil is separated from the mixture together with the volatile working fluid.

13. Composition according to claim 12, wherein the volatile working fluid consists of at least one of a fatty acid ester, a fatty ester amide, a free fatty acid, bio-diesel and a hydrocarbon or any combinations thereof.

14. Composition according to claim 12, wherein the volatile working fluid comprises at least one fatty acid ester composed of C10-C22 fatty acids and C1-C4 alcohols, or a combination of two or more fatty acid esters each composed of C10-C22 fatty acids and C1-C4 alcohols.

15. Composition according to claim 12, wherein the volatile working fluid is essentially equally or less volatile than the environmental pollutants that are to be separated from the fat or oil mixture.

16. Composition according to claim 12, wherein the ratio (volatile working fluid):(fat or oil mixture) is about 1:100 to 15:100.

17. Composition according to claim 1, wherein said fat or oil mixture is a vegetable oil or fat; a microbial oil or fat;
or animal oil or fat, such as a marine oil or fat, e.g. a marine oil obtained from fish or sea mammals.

18. Composition according to claim 1, wherein said fat or oil mixture contains an amount of free fatty acids that is sufficient to act as an internal volatile working fluid.

19. Composition according to claim 1, wherein said composition is a feed composition.

20. Composition according to claim 19, wherein said feed composition is a fish feed composition.

21. Composition according to claim 4, wherein said composition is a health supplement.

22. Composition according to claim 4, wherein said at least one easily oxidised component is selected from the group consisting of astaxanthin, tocopherols, tocotrienols, ubiquinones, lutein, polyunsaturated fatty acids and derivatives of polyunsaturated fatty acids or any combination thereof.

23. Composition according to claim 1, wherein said environmental pollutants are selected from the group consisting of growth inhibiting factors, substances which accelerate sexual maturation, substances which promote oxidative damage and substances which promotes oxidation of easily oxidised components.

24. Composition according to claim 2, wherein said composition reduces the tendency of an animal to enter sexual maturation beyond what is achieved when using a composition comprising said oil or fat mixture that has not been subjected to said processing steps.

25. Composition according to claim 3, wherein said composition reduces or prevents oxidative damage in an animal beyond what is achieved when using a composition comprising said oil or fat mixture that has not been subjected to said processing steps.

26. Composition according to claim 3, wherein said composition reduces or prevents oxidative stress in an animal beyond what is achieved when using a composition comprising said oil or fat mixture that has not been subjected to said processing steps.

27. Composition according to claim 4, for reducing oxidation of the at least one easily oxidised component during production and storage compared with what is the case for a composition comprising said oil or fat mixture that has not been subjected to said processing steps.

28. Composition according to claim 4, for increasing the utilization of the at least one easily oxidised component compared with what is the case for a composition comprising said oil or fat mixture that has not been subjected to said processing steps.

29. Composition according to claim 20, wherein the fillet from fish fed a composition comprising the processed oil or fat mixture has a pre-rigor gaping score that is less than 0.4.

30. Composition according to claim 19, wherein the utilization of omega-3 fatty acids in said feed is increased compared to a feed comprising said oil or fat mixture that has not been subjected to said processing steps.

31. Composition according to claim 19, wherein the feed conversion ratio is significantly reduced as compared to feed comprising said oil or fat mixture that has not been subjected to said processing steps.
